(19) United States
(12) Patent Application Publication
(10) Pub. No.: US 2025/0201425 A1
(43) Pub. Date: Jun. 19, 2025

(54) METHOD FOR MEASURING THE SURFACE OF THE SCALP TO ASSESS BALD AREAS (RECIPIENT AREAS), AREAS WITH THINNING HAIR, AND AREAS WITH THICK HAIR (DONOR AREAS)

(71) Applicants: CARLO GERALDO FRAGA DE CASTRO, Vitória (BR); FREDERICO PITTELLA SILVA, Juiz de Fora (BR); HAIR RESTORATION SCIENCE LTDA, Linhares (BR)

(72) Inventor: FELIPE A. PITTELLA SILVA, Vitória (BR)

(21) Appl. No.: 18/982,020

(22) Filed: Dec. 16, 2024

Related U.S. Application Data
(60) Provisional application No. 63/611,273, filed on Dec. 18, 2023.

(51) Int. Cl. G16H 50/50; A61B 5/00; G06T 7/00; G06T 7/62; G06T 17/20; G16H 30/40

(52) U.S. Cl. CPC G16H 50/50; G06T 7/0012; G06T 7/62; G06T 17/20; G16H 30/40; A61B 5/446; G06T 2207/10028; G06T 2207/20104; G06T 2207/30088; G06T 2207/30242; G06T 2210/56

(57) ABSTRACT

The present invention relates to a Method for Measuring the surface of the scalp for assessing bald areas (recipient areas), areas with thinning hair, and areas with thick hair (donor areas), using the counting of micropolygons in 3D models and/or 3D meshes created by technologies that generate full-scale 3D models. Additionally, the present invention pertains to a Method for Mapping the surface of the scalp to measure bald areas (recipient areas), areas with thinning hair, and areas with thick hair (donor areas). The invention also relates to a corresponding system and a computer program for these purposes

### CROSS-REFERENCE TO RELATED APPLICATIONS

This patent application claims the benefit of priority from U.S. Provisional Patent Application No. 63/611,273, filed on Dec. 18, 2023. The aforementioned application is fully incorporated herein by reference for multiple purposes.

### FIELD

The present invention pertains to the field of trichology and medical imaging and relates to a Method for Measuring the surface of the scalp to assess bald areas (recipient areas), areas with thinning hair, and areas with thick hair (donor areas), using the counting of micropolygons in 3D models and/or 3D meshes created by technologies that generate full-scale 3D models. Additionally, the present invention pertains to a Method for Mapping the surface of the scalp to measure bald areas (recipient areas), areas with thinning hair, and areas with thick hair (donor areas). The invention also relates to a corresponding system and a computer program for these purposes.

### BACKGROUND

Hair transplantation is a surgical procedure designed to treat androgenetic alopecia or other forms of permanent hair loss by transferring intact and healthy follicular units from a donor region to a recipient region on the patient's scalp, characterized by the absence or thinning of hair.

This procedure involves the removal of grafts or follicular units, consisting of groups of 1 (one) to 4 (four) hair follicles, commonly harvested from the sides and back of the scalp, areas with genetic attributes resistant to alopecia. These grafts are then meticulously implanted in the bald or low-density follicular recipient areas.

For a hair transplant, it is recommended that the patient consult a medical specialist in trichology or hair surgery for a thorough evaluation of the affected areas and an accurate diagnosis of the alopecia's etiology, in order to prescribe the most suitable transplant technique for the case in question.

Generally, the initial assessment involves a detailed physical examination to evaluate the donor area and determine the number of sessions needed for adequate coverage of the recipient area. This analysis is typically conducted both clinically and instrumentally, utilizing imaging generation and analysis tools such as a trichoscope. The examination conducted by the physician can provide information about the patient's hair health, the expected progression of alopecia, as well as analyze the viability of the donor area and assess the overall health of the scalp.

Based on this assessment, the most appropriate therapeutic or surgical strategy is discussed, primarily taking into account the type and stage of alopecia.

It is imperative to highlight that the choice of the hair transplant technique to be performed is currently based on the classification of the degree of baldness, obtained through the previously outlined examination.

The degree of baldness, qualitatively determined by scales such as the Norwood-Hamilton Scale, is a globally recognized metric for assessing and categorizing the progression of alopecia, providing a qualitative analysis of the scalp.

However, when adopting assessment practices that focus on a qualitative analysis of the scalp, challenges arise related to the difficulties in accurately measuring the areas affected by alopecia.

Thus, despite the existence of qualitative assessment methods for the scalp, there is currently no efficient method to calculate, quantitatively and accurately, the bald or thinning surface. Traditional methods, such as rulers and measuring tapes, prove to be inadequate. Therefore, a methodology is needed that can quantitatively assess the bald or thinning surface to be subjected to hair transplantation.

It is crucial to emphasize that existing methods for assessing the alopecic surface are unable to provide, quantitatively, the exact dimension of the scalp that requires surgical intervention, influencing the efficiency of the transplant, associated costs, and the time required for the procedure.

Therefore, there is a need to develop a methodology that can accurately measure the surfaces that will be subject to surgical procedures, providing ease and precision in measuring alopecic areas, as well as allowing the evaluation of cranial curvature and the measurement of areas with varying hair density.

The present invention aims to overcome the aforementioned disadvantages, given the lack of a solution in the market that addresses these challenges. Therefore, there is a need in the art to develop a method for mapping the scalp surface for measuring bald areas (recipient areas), areas with thinning hair, and areas with thick hair (donor areas).

The proposed method will be able to provide ease and precision in measuring bald areas, as well as cranial curvature, filling an existing gap.

Unlike existing qualitative classifications, which do not take into account the measurement of bald areas, the measurement proposed by the present invention will be able to accurately determine the total area of the bald surface and, therefore, enable appropriate surgical intervention.

The invention will also help in defining the medical approach, providing greater precision in the execution of surgical procedures and, as a result, leading to better outcomes and patient satisfaction.

This method not only revitalizes the approach to scalp mapping but also infuses technical precision and visualization capabilities that are crucial for optimizing the outcomes of hair transplant procedures, ensuring effectiveness and aesthetics in follicular redistribution.

Thus, the geometry of the scalp, characterized by pronounced curvatures and irregular surfaces, poses challenges for traditional area measurement methods, which often suffer from distortions caused by these peculiarities.

The proposed method, however, compensates for these irregularities in a precise and efficient manner. The detailed subdivision, enabled by the high density of micropolygons, allows for the accurate mapping of every minor variation on the surface, with the curvature and concavity of each region being faithfully reflected in the shape and orientation of the micropolygons. Furthermore, automatic error correction, achieved through the individualized calculation of the areas of micropolygons and the subsequent summation of these areas, significantly reduces distortions and prevents inaccurate approximations. The computer program also applies real-time dynamic adjustments, allowing adaptation to more complex regions, ensuring that the measurement maintains a high level of precision under all circumstances.

All these objectives, features, and advantages of the present invention will become readily apparent after a deeper reading of the detailed description of the preferred embodiments, as illustrated in the attached figures.

### SUMMARY

The present invention describes a **Method for Measuring** the surface of the scalp to assess bald areas (recipient areas), areas with thinning hair, and areas with thick hair (donor areas), using the counting of micropolygons in 3D models and/or 3D meshes created by technologies that generate full-scale 3D models.

Additionally, the present invention relates to a **Method for Mapping** the surface of the scalp to measure bald areas (recipient areas), areas with thinning hair, and areas with thick hair (donor areas).

The application also refers to a corresponding system and a computer program for these purposes.

One objective of the present invention is to provide methods, a system, and a computer program capable of accurately measuring the surfaces that will undergo surgical procedures, offering ease and precision in measuring alopecic areas, in addition to enabling the evaluation of cranial curvature and measuring areas with varying hair density.

Another objective of the present invention is to provide ease and precision in measuring bald areas, as well as cranial curvature, addressing a gap currently existing.

Another objective of the present invention is to assist in defining the medical procedure, ensuring greater precision in performing surgical procedures and, as a result, providing better outcomes and patient satisfaction.

Another objective of the present invention is to provide a method capable of determining the size of the bald area, in order to define the appropriate surgical approach. With more precise measurement of bald areas, it is possible to make a more accurate classification of baldness and improve the definition of the surgical approach in hair transplant procedures.

Moreover, another objective of the present invention is to offer a series of benefits with the proposed methods, system, and computer program, such as greater simplicity in measuring bald areas, not being affected by cranial curvature or hair characteristics. The resulting 3D model also aids in communication with the patient, allowing for better visualization and understanding of the bald areas to be treated. It is also an objective of the present invention to provide an innovative and precise method for measuring the surface of bald areas on the scalp using technologies equipped with mechanisms for capturing three-dimensional information, offering a more accurate and precise measurement. This contributes to a better classification of baldness, a more precise definition of the surgical approach, and a fairer quantification of the services provided.

The unique measurement method, using the counting of micropolygons in 3D models and/or 3D meshes created by technologies equipped with mechanisms for capturing three-dimensional information, stands out as a strong point of this method since, regardless of the application used, this technology is capable of calculating the area of baldness, taking into account the patient's scalp curvature.

Although the invention has been described and exemplified based on three preferred embodiments, it is important to emphasize that there are possible variations that fall within the scope of the present invention.

In conclusion, the methods, system, and computer program described in this invention represent a significant advancement in the precise measurement of bald areas on the scalp. By employing technologies equipped with mechanisms for capturing three-dimensional information, such as TrueDepth, Structured Light, LIDAR, Time-of-Flight (ToF) Sensors, Photogrammetry, and Stereoscopic Vision, it is possible to achieve greater accuracy in determining the size of the bald area, thus improving the efficiency and outcomes of hair transplant procedures. These methods offer simplicity, precision, and clear visualization of bald areas for the patient, providing a personalized and effective approach to treating baldness.

### BRIEF DESCRIPTION OF THE DRAWINGS

These and other features, aspects, and advantages of the present invention will be better understood in relation to the following description and accompanying drawings, using at least two of the preferred technologies from one embodiment of the present invention, namely LiDAR (Light Detection and Ranging) technology and Photogrammetry technology. These figures are purely schematic, and their proportions and dimensions may not correspond to reality, as they are intended solely to describe the invention in a didactic manner. Therefore, the figures are not limiting, except as defined by the subsequent claims. One embodiment of the invention is hereinafter described with reference to the accompanying drawings, in which:

FIG. 1 shows an illustrative image of a doctor turning on their mobile electronic device, equipped with LiDAR technology, and accessing the 3D image processing application to initiate the measurement process of the patient's bald area, according to various embodiments described herein,

FIG. 2 illustrates the doctor's visual perception, via a touchscreen display, of the activated 3D image processing application, ready to use the camera to scan the patient's scalp surface, ensuring that the mapping and analysis functionalities are technically aligned to capture and process data with maximum efficiency and precision, according to various embodiments described herein,

FIG. 3 illustrates the formation of the 3D point cloud, created by the emission of laser pulses from the technology, which accurately maps the scalp, forming a conglomerate of three-dimensional data that will be instrumental in creating a faithful graphical representation of the areas of interest, according to various embodiments described herein,

FIG. 4 illustrates the 3D mesh (“3D MESH”) generated from the 3D point cloud, through the delimitation and coloring of the scanned scalp region, providing a more tangible and manipulable visualization for analysis and surgical planning, according to various embodiments described herein,

FIG. 5 illustrates the phase of measuring the 3D mesh area (“3D MESH”), where specific algorithms calculate the area based on the points and planes defined in the mesh, offering essential quantitative data for proper planning of the hair transplant, according to various embodiments described herein,

FIG. 6 illustrates the 3D model generated by the application, where the previously acquired and processed information is transformed into a tangible three-dimensional model, providing a robust visual tool for both professional analysis and doctor-patient communication, according to various embodiments described herein, and

FIG. 7 is a process flow of an exemplary embodiment of a method for mapping the scalp surface for measuring bald areas (receiver areas), areas with thinning hair, areas with thick hair (donor areas).

FIG. 08 is a process flow of an exemplary embodiment of a Method for Measuring the surface of the scalp to assess bald areas (recipient areas), areas with thinning hair, and areas with thick hair (donor areas), using the counting of micropolygons in 3D models and/or 3D meshes created by technologies that generate full-scale 3D models.

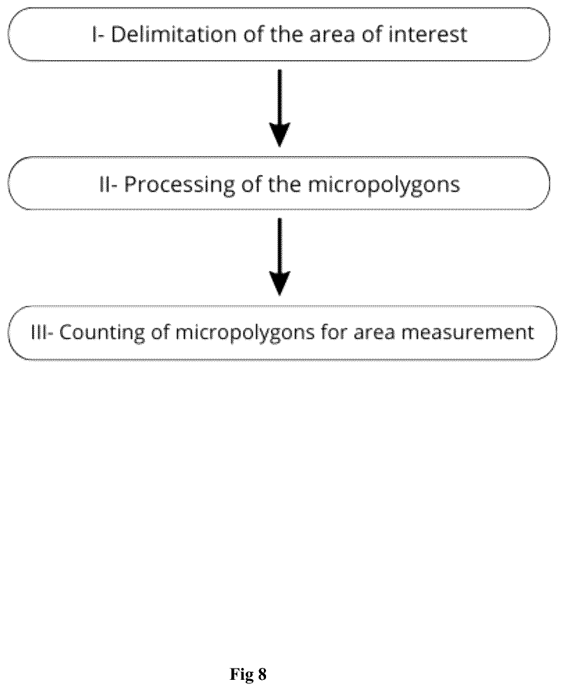

Each of the figures, and the technical elements incorporated within them, represents a crucial step in the advancement proposed by this invention, providing an innovative and highly precise approach to the mapping and analysis of bald areas and, consequently, to the enhancement of hair transplant techniques and results.

Other aspects and features of the example embodiments described herein will become apparent from the following description, along with the accompanying drawings.

### DETAILED DESCRIPTION

The following description presents the preferred mode of some of the embodiments of the present invention. It is clear from this description that the invention is not limited to these illustrated embodiments, but also includes a variety of modifications and embodiments of the same. Therefore, this description should be seen as illustrative and not limiting. Although the invention is susceptible to various modifications and alternative constructions, it is important to emphasize that there is no intention to limit the invention to the specific form disclosed, but rather to encompass all modifications, alternative constructions, and equivalents within the spirit and scope of the invention. In any embodiment described herein, the open-ended terms “comprising,” “comprises,” and similar terms (which are synonyms for “including,” “having,” and “characterized by”) may be replaced with the partially closed phrases “consisting essentially of,” “consists essentially of,” and similar terms, or with the fully closed phrases “consisting of,” “consists of,” and similar terms.

As used herein, the singular forms “a,” “an,” “the” refer to both the singular and the plural, unless expressly stated to refer only to the singular.

The systems and methods discussed in this document can be implemented using either hardware, software, or a combination of both. Such implementations can be carried out in computer programs running on programmable computers. Each of these computers is equipped with at least one processor, a data storage system, which may include volatile memory, non-volatile memory, or a combination of both, and at least one network communication interface.

By way of example, and without limiting the possibilities, programmable computers include a range of devices such as servers, network devices, embedded devices, computer expansion modules, personal computers, laptops, personal digital assistants, mobile phones, smartphones, tablets, wireless devices, or any other computing device that can be configured to execute the methods described herein. In a first aspect, the invention relates to a **System for Measuring** the surface of the scalp to assess bald areas (recipient areas), areas with thinning hair, and areas with thick hair (donor areas), using the counting of micropolygons in 3D models and/or 3D meshes created by technologies that generate full-scale 3D models, the system comprising at least one camera hardware equipped with at least one technology for capturing three-dimensional information, at least one electronic device, at least one measurement application or software installed on the electronic device, and a processor configured to execute any of the methods described in the present invention.

In a preferred embodiment, the camera hardware is equipped with at least one technology for capturing three-dimensional information.

In an ideal embodiment, the technologies for capturing three-dimensional information are preferably TrueDepth, Structured Light, LiDAR, Time-of-Flight (ToF) Sensors, Photogrammetry, and Stereoscopic Vision.

In one of the ideal applications of this invention, the technology equipped with three-dimensional information capture mechanisms used is the TrueDepth technology developed by Apple Inc.

The TrueDepth system consists of a combination of hardware and software working together to enable facial recognition and 3D image capture. The technology is based on a point-projection sensor, which is the central component of the system, emitting an infrared light point pattern on the user's face. These points are projected onto a 3D grid to create an accurate map of the facial topography. Meanwhile, the infrared camera sensor captures images of the face, which are combined with the light point map to create a detailed three-dimensional representation.

In another ideal application of this invention, the technology equipped with three-dimensional information capture mechanisms used is Structured Light technology, which utilizes light patterns projected onto an object to measure its shape and depth. The system employs a specific light structure, usually from a high-definition projector or a meticulously organized LED matrix. This light, when projected, forms specific and measurable patterns onto the target object, in this case, the surface of the scalp and/or hair follicles.

The Structured Light technology used here is calibrated to project light patterns, which, once distorted by the three-dimensional surface of the object being analyzed, are subsequently captured and analyzed by a camera, or more precisely, a high-resolution optical capture device. This device is equipped with algorithms that analyze the deformations in the light patterns, enabling the deduction of the three-dimensional shape of the object under study.

The projected light patterns and the resulting deformations are analyzed using a combination of computer vision algorithms and machine learning, enabling a meticulous and detailed assessment of the scalp surface and a precise 3D digital representation. This representation, in turn, is essential for planning hair transplant procedures, providing a personalized and strategically informed approach for each patient.

In another ideal application of this invention, the technology equipped with three-dimensional information capture mechanisms used is LiDAR technology, which stands for Light Detection and Ranging, a remote sensing system that uses laser pulses to measure the distance between the sensor and an object or surface. The basic principle of LiDAR involves emitting a laser light pulse and measuring the time it takes for the pulse to return after hitting the object, enabling precise calculation of the distance to it.

In detail, the process involves emitting a laser light pulse, which travels until it hits the object and is reflected back to the LiDAR sensor. The return time of this pulse is meticulously calculated, allowing for the exact determination of the distance between the sensor and the object.

In another ideal application of this invention, the technology equipped with three-dimensional information capture mechanisms used is Time-of-Flight (ToF) Sensors technology, which is used to measure distances by calculating the time it takes for a light pulse to travel to an object and return to the sensor. The ToF Sensors operate by emitting a light pulse, typically in the infrared range, directed at the target object, and then carefully recording the time it takes for the light to return after reflecting off the object.

In another ideal application of this invention, the technology equipped with three-dimensional information capture mechanisms used is Photogrammetry technology, which involves capturing multiple images of an object or environment from different angles and then processing them to create a 3D model. Initially, the system analyzes the captured images, identifying singularized reference points that are meticulously correlated across the various images to compute the three-dimensional spatial coordinates of the object or surface under study.

In other ideal applications of this invention, the technology equipped with three-dimensional information capture mechanisms used is Stereoscopic Vision technology, which involves using two cameras or sensors positioned slightly differently to capture images from slightly different angles. The technology enables depth perception based on the perspective differences between the images captured by each “eye.” It is a technique used in various applications, such as 3D cinema and virtual reality. By comparing the differences between the images, it is possible to infer information about the depth of the object.

In a preferred application, the electronic device is preferably a mobile phone or tablet. In another preferred application, the electronic device may be any electronic device intended for mapping and 3D reconstruction, equipped with technologies that create real-size 3D models.

In a preferred application, the image processing and measurement application or software installed on the electronic device is configured to:
a) Capture three-dimensional information from the scalp;
b) Receive the digital image data collected by the technology equipped with three-dimensional information capture mechanisms;
c) Analyze the captured images, identifying and correlating reference points that are linked across the various images to compute the three-dimensional spatial coordinates of the scalp;
d) Construct a 3D point cloud that accurately represents the geometry and topography of the scanned scalp;
e) Delimit, color, and automatically register the region, generating a 3D mesh that displays detailed and continuous visualizations of the scanned scalp surface;
f) Create, from the 3D mesh, a continuous and detailed visual representation of the scanned scalp surface;
g) Measure the area of the scalp through tactile interaction in the desired region;
h) Display, for viewing through the electronic device, the simulated image of the scalp.

In a preferred application, the processor is configured to execute any of the methods described in the present invention.

In a preferred application, the processor is a data storage system, which may include volatile memory, non-volatile memory, or a combination of both, and at least one network communication interface.

In another aspect, the present invention refers to a Method for Measuring the Scalp Surface, the method comprising the counting of micropolygons for measuring bald areas (receiver areas), areas with fine hair, areas with thick hair (donor areas), through the measurement of the surface of 3D models and/or 3D meshes created by technologies that generate real-size 3D models.

In an ideal application, the method uses 3D models and/or 3D meshes created by technologies equipped with real-size three-dimensional information capture mechanisms.

In an ideal application, the 3D model and/or 3D mesh of the scalp surface is generated from a detailed scan performed by an electronic device. The scanning can be carried out by any electronic device equipped with technology having real-size three-dimensional information capture mechanisms.

In an ideal application, the technologies equipped with real-size three-dimensional information capture mechanisms are preferably TrueDepth, Structured Light, LiDAR, Time-of-Flight (ToF) Sensors, Photogrammetry, and Stereoscopic Vision.

In an ideal application, the app will scan the patient's scalp surface, obtaining data that is combined with images captured by a traditional camera, creating a three-dimensional point cloud that accurately represents the anatomical characteristics of the scalp.

From this point cloud, a 3D mesh is generated through the application of advanced processing algorithms that connect adjacent points and form triangular or polygonal faces. This mesh, composed of vertices, edges, and micropolygons, continuously and accurately represents the scalp surface, enabling precise geometric analysis.

The term “micropolygon” refers to small subdivisions of the 3D mesh created to capture the fine details of the scalp surface, allowing for area calculation with high precision. By subdividing the mesh into micropolygons, the method eliminates errors associated with coarse approximations or the use of larger polygons, which are common in traditional techniques. Additionally, the high density of micropolygons in the 3D mesh allows for the capture of even the smallest topographic nuances, ensuring detailed and accurate measurement.

In an ideal application, from the acquisition of the 3D model and/or 3D Mesh, using technologies with three-dimensional information capture mechanisms, the measurement method, the subject of this invention, will be carried out, comprising the following steps:

### I—Delimitation of the Area of Interest:

The delimitation of the area of interest is performed by the operator through an interactive interface on the electronic device, directly on the virtual surface of the 3D model or 3D mesh, using any interactive interface designed to capture multiple input modalities.

The available interfaces for interaction include, but are not limited to: tactile gestures, where the operator can use taps or swipes on touch-sensitive device screens to trace contours around the desired area or select specific regions, such as bald, recipient, donor, or thinning hair areas; pointing devices, such as mice, stylus pens, and other peripherals, allowing for more precise interaction, especially in regions with complex geometries; and gesture and voice recognition, enabling remote control of the system through gestural or vocal commands, enhancing versatility and facilitating operation in various clinical environments and scenarios.

II—Processing of micropolygons:

After the delimitation of the area of interest, the system processes the micropolygons contained within the selected region, regardless of the interaction method used, performing the calculation of their areas with high precision. The software identifies the micropolygons that are fully or partially included in the demarcated area, and in the case of micropolygons that intersect the boundary of the region of interest, it applies techniques, preferably clipping, to adjust the calculation, considering exclusively the relevant portion within the defined boundaries.

### III—Counting of Micropolygons for Area Measurement:

After the delimitation of the area of interest and the data processing, the system automatically calculates the total area by summing the individual areas of the micropolygons that make up the selected region.

Each micropolygon is treated as an individual geometric unit, which may take the form of triangles or regular and irregular polygons. Its area is calculated precisely using specialized computational geometry algorithms, based on the vertices and edges that define its configuration.

For micropolygons that are only partially contained within the area of interest, intersection algorithms are applied to determine the exact fraction of the area to be included in the calculation. In the case of triangles, the Heron's formula or the basic area calculation formula (base×height÷2) is preferably used. For more complex polygons, such as quadrilaterals or irregular shapes, the areas can be subdivided into triangles or determined directly through methods like Gauss's area formula, ensuring accuracy even on surfaces with greater geometric complexity.

The total area determination is performed by the cumulative sum of the individual areas of the micropolygons that make up the selected region. This method ensures a high level of precision, as it is capable of compensating for geometric distortions caused by the curvature or irregularity of the scalp surface.

Each micropolygon in the demarcated region is calculated individually, with their areas being summed to obtain the final area precisely. This process is fully automated through specialized triangulation and numerical integration algorithms, ensuring fast and accurate execution, even in regions with thousands of micropolygons.

Due to the small size of the micropolygons, their areas faithfully represent even the smallest variations in the topology of the scanned surface, ensuring high resolution in the results obtained.

In another aspect, the present invention relates to a Method for Mapping the Surface of the Scalp for Measuring Bald Areas (receptor areas), Areas with Thinning Hair, and Areas with Thick Hair (donor areas), comprising the following steps: Device Activation and Scalp Scanning, Data Capture and 3D Point Cloud Formation, Generation of the 3D Mesh (“3D MESH”), and Area Measurement.

In an ideal application, the method consists of the use of technologies equipped with mechanisms for capturing three-dimensional information, which will be used to generate a 3D mesh (“3D MESH”) that will serve as the foundation for rendering the patient's 3D scalp model. This model will enable the measurement of bald areas (receptor areas), areas with thinning hair, and areas with thick hair (donor areas). The 3D mesh not only consolidates visual data but also provides a volumetric understanding of the scalp regions, thus allowing for more rigorous and personalized planning for hair implant procedures.

In an ideal application, once the 3D model and/or 3D Mesh is obtained using technologies equipped with mechanisms for capturing three-dimensional information, the measurement method of the present invention will be carried out, comprising the following steps: Device Activation and Scalp Scanning; Data Capture and 3D Point Cloud Formation; Generation of the 3D Mesh (“3D MESH”); Area Measurement.

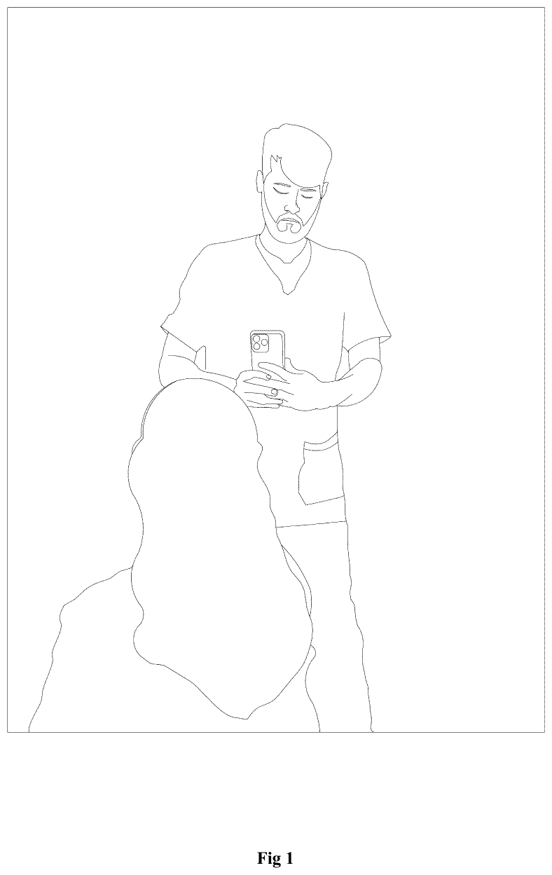

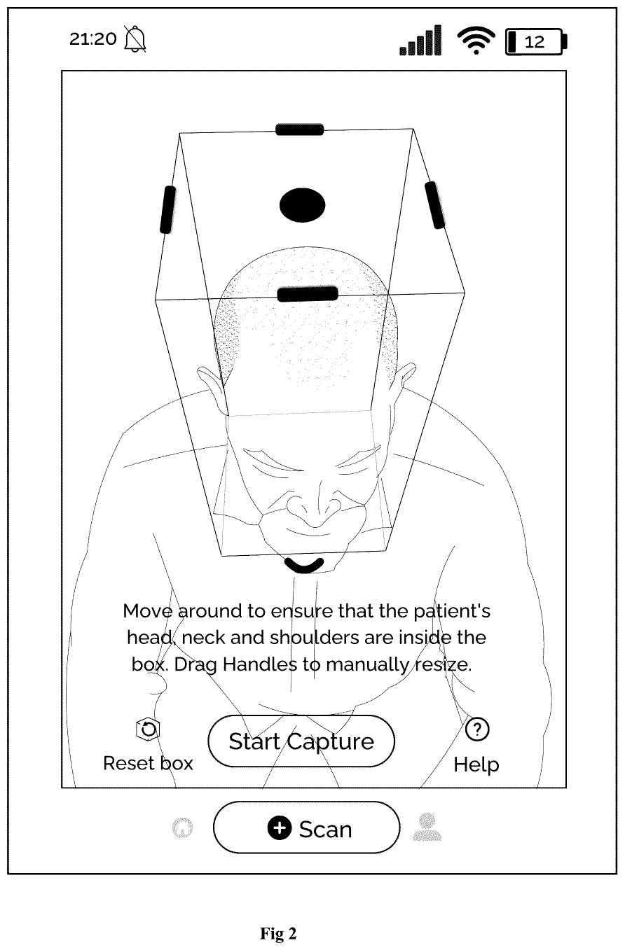

In an ideal application, the Device Activation and Scalp Scanning step involves the system operator turning on the electronic device (cell phone or tablet) equipped with one of the technologies that create real-scale 3D models, opening the specialized measurement application (FIG. 01). With the application open (FIG. 02), the operator, using the device's camera, will position the electronic device in such a way that the patient's scalp surface is within the field of the utilized technology.

With the application still open (FIG. 02), the operator, using the device's camera, proceeds to the detailed scanning of the entire scalp surface of the patient who wishes to perform the measurement.

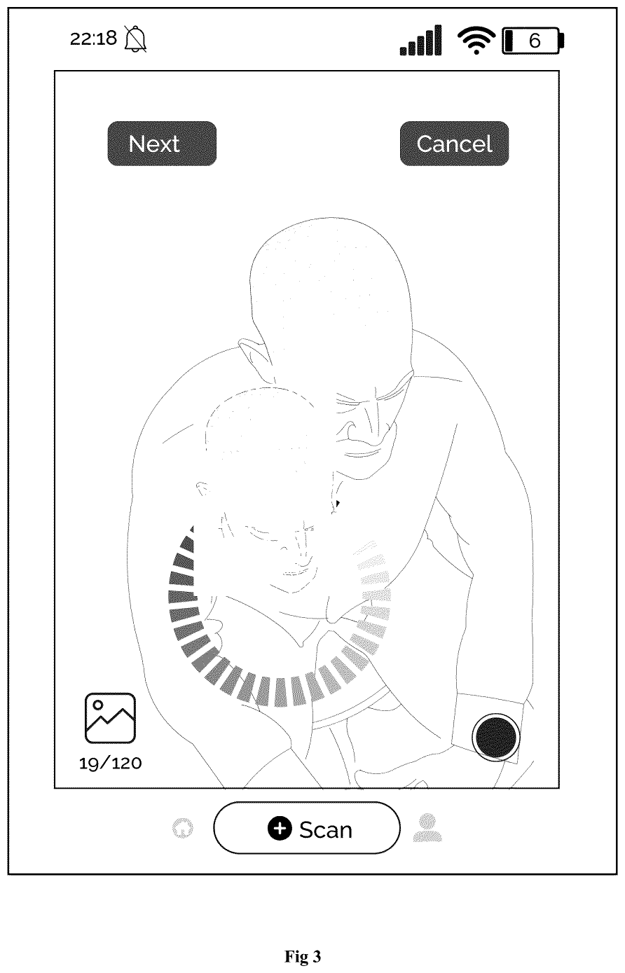

In an ideal application, the Data Capture and 3D Point Cloud Formation step involves the creation of a 3D point cloud (FIG. 03) that accurately represents the geometry and topography of the scanned scalp.

The data in the 3D point cloud are a spatial representation of the collected data points and can be used to depict the 3D morphology of the patient's scalp.

In an ideal application, the 3D Mesh Generation (“3D MESH”) step involves the operator marking points through the application, delimiting, painting, and automatically recording the region, thus generating a more detailed mesh model of the scalp, created based on data from the technology that creates 3D models.

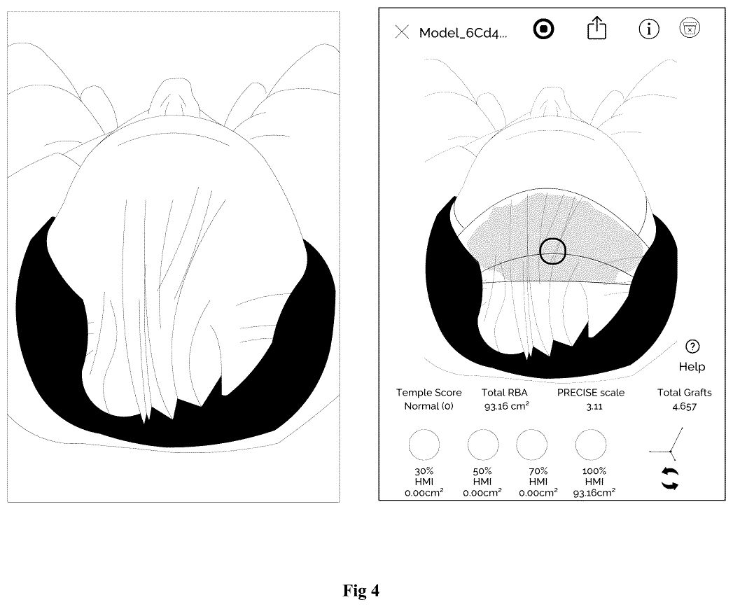

This mesh is known as the 3D mesh (“3D MESH”) (FIG. 04) and will be generated from the initial point cloud, where computational algorithms and image processing techniques are applied to connect adjacent points and form triangular or polygonal faces. These connected faces create a continuous and detailed visual representation of the scanned scalp surface.

The 3D mesh (“3D MESH”) consists of vertices (points in the point cloud), and edges and faces that connect these vertices, generating a coherent and three-dimensional spatial representation. The density and detail level of the 3D mesh (“3D MESH”) may vary according to the resolution of the data derived from the technology used to create 3D models or other listed technologies and the algorithms employed in the mesh generation process, allowing adjustments to the granularity and accuracy of the model.

In an ideal application, the measurement step involves using the Scalp Surface Measurement Method for measuring bald areas (recipient areas), areas with fine hair, areas with thick hair (donor areas), utilizing the counting of micropolygons in 3D models and/or 3D meshes created by technologies that generate real-size 3D models, as described earlier in this invention.

In an additional aspect, the invention refers to a computer program product comprising a computer program with instructions organized to, when executed by a computer, perform at least part of the method described above. The computer program product may be incorporated into a computer-readable medium, such as a hard drive, solid-state memory, flash memory, etc., and may be non-writable or writable. The computer program product may, for example, include a known algorithm for image recognition and/or image measurements. These can be used to process and analyze the image data provided by camera and optical acquisition means.

In order to avoid repetitions in this description, it is understood that the features previously described for the aspects related to the system and/or methods of the invention are also applicable to the aspect referring to the computer program product, and vice versa. Thus, these features should be considered as disclosed and subject to claims for both aspects, for the computer program product as well as the system and the method. The same applies to the aspects related to the system and the method among themselves.

### Description of Preferred Embodiments

The mapping method, one of the objects of the present invention, will be described below, using as reference three of the preferred technologies previously described in the body of the present invention. It is imperative to emphasize that, although three technologies are preferably addressed for subsequent exemplification, the essence and applicability of the method remain intact and fully viable when applied to other three-dimensional measurement technologies, thus preserving the integrity and objectivity inherent in the invention described herein.

Other characteristics and advantages of the present application will become apparent from the following detailed description. However, it should be understood that the detailed description and specific examples, while indicating implementations of the application, are provided merely for illustration, and the scope of the claims should not be limited by these implementations but should be given the broadest interpretation consistent with the description as a whole.

Reference is now made to Figures FIG. 01 to FIG. 06, which show an example of the implementation of a method for mapping the scalp surface to measure bald areas (recipient areas), areas with thinning hair, and areas with thick hair (donor areas) according to at least some of the implementations, using the LiDAR three-dimensional data capture technology.

In one of the ideal applications of the present invention, the measurement of bald areas (recipient areas), areas with thinning hair, and areas with thick hair (donor areas) will be carried out through a system that includes the use of a mobile application or other image processing software for 3D models, properly downloaded onto a mobile phone, tablet, or any electronic device designed for 3D mapping and reconstruction, equipped with LiDAR technology, which will capture images of the scalp.

In an ideal application, the method of the present invention consists of using LiDAR technology to measure bald areas (recipient areas), areas with thinning hair, and areas with thick hair (donor areas), by measuring the surface of 3D models and/or 3D meshes (“3D MESH”) created by these technologies or by other similar technologies that generate real-size 3D models, comprising the following steps:

### I—Activation of the Device and Scalp Scanning:

To perform the mapping and measurement of the scalp, the system operator will turn on the electronic device (mobile phone or tablet) equipped with LiDAR technology and open the specialized measurement application (FIG. 01). With the application open (FIG. 02), the operator, using the device's camera, will position the electronic device such that the surface of the patient's scalp is within the LiDAR's field of view. With the application still open (FIG. 02), the operator, using the device's camera, proceeds to the detailed scanning of the entire surface of the patient's scalp to be measured.

### II—Data Capture and Formation of the 3D Point Cloud:

At this point, the LiDAR will emit laser pulses that, when interacting with the scalp surface, are reflected back to the integrated sensor. The temporal response of the reflected pulses is processed, forming a 3D point cloud (FIG. 03) that accurately represents the geometry and topography of the scanned scalp.

The data from the 3D point cloud are a spatial representation of the collected data points and can be used to represent the 3D morphology of the patient's scalp.

### III—Generation of the 3D Mesh (“3D MESH”);

As the points are marked by the operator, the application automatically delineates, paints, and registers the region, generating a more detailed mesh model of the head, created based on LiDAR data, as used in the example.

This mesh is known as the 3D mesh (“3D MESH”) (FIG. 04) and will be generated from the initial point cloud, where computational algorithms and image processing techniques are applied to connect adjacent points and form triangular or polygonal faces. These connected faces create a continuous and detailed visual representation of the scanned scalp surface.

The 3D mesh (“3D MESH”) is composed of vertices (points in the point cloud), edges, and faces that interconnect these vertices, generating a coherent and three-dimensional spatial representation. The density and level of detail of the 3D mesh (“3D MESH”) may vary according to the resolution of the data derived from LiDAR or other listed technologies, and the algorithms used in the mesh generation process, allowing adjustments to the granularity and accuracy of the model.

### IV-Area Measurement:

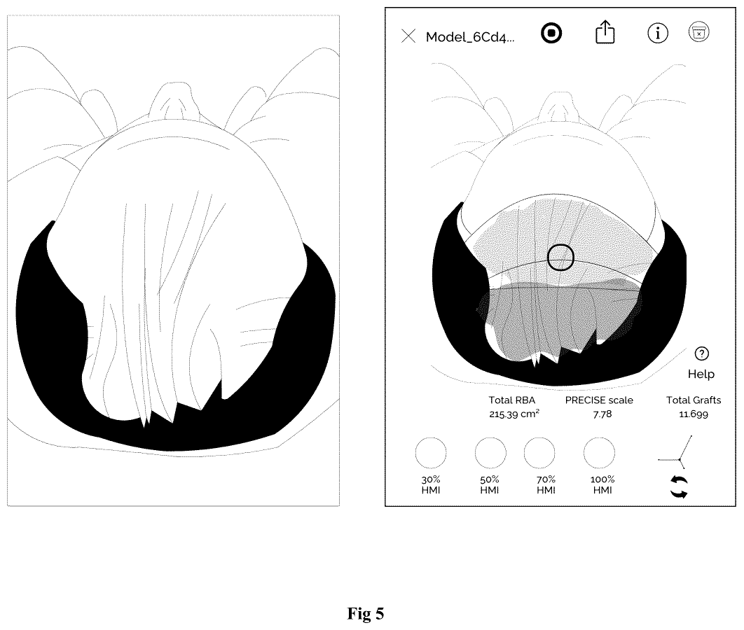

The area measurement (FIG. 05) is performed through tactile interaction in the desired region. The operator slides their finger over the 3D model on the application's touch interface so that the corresponding area of the 3D mesh is measured precisely.

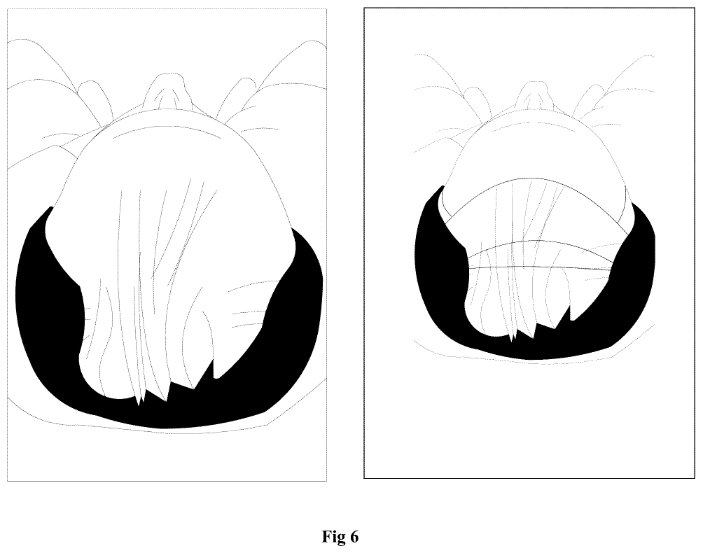

The mesh is composed of micropolygons, enabling precise calculation of the surface area, even in regions with complex or irregular curvature. The measurement is carried out by the application through the summation of the areas of the micropolygons in the demarcated regions. This measurement method mitigates distortions caused by the irregularity and curvature of the scalp surface, providing a high degree of precision in measuring bald areas, as the 3D model (FIG. 06) compensates for the distortions and irregularities of the scalp surface.

As the distances in the model are mapped onto the 3D mesh, the area is calculated on the surface of the head, ensuring an authentic measurement of the hairless areas, thus representing a precise and clinically valuable approach to quantifying bald regions or areas for potential hair implants.

In another ideal application of the present invention, the measurement of bald areas (receptive areas), areas with thinning hair, and areas with thick hair (donor areas) will be carried out through the use of a mobile application or other image processing software for 3D models, properly downloaded onto a smartphone, tablet, or any electronic device designed for 3D mapping and reconstruction, equipped with TrueDepth technology, which will capture images of the scalp.

In an ideal application, the method of the present invention consists of using TrueDepth technology to measure bald areas (receptive areas), areas with thinning hair, and areas with thick hair (donor areas) by measuring the surface of 3D models and/or 3D meshes (“3D MESH”) created by these technologies or other similar technologies that create full-size 3D models. The method includes the following steps:

### I—Device Activation and Scalp Scanning:

To perform the measurement, the operator will turn on the electronic device (smartphone or tablet) equipped with TrueDepth technology and open the specialized measurement application/software. Subsequently, with the application/software open, the operator, using the device's camera, will position the electronic device so that the surface of the patient's scalp is within the TrueDepth field of view.

While the application is still open (FIG. 02), the operator, using the device's camera, proceeds with the detailed scanning of the entire surface of the patient's scalp that requires measurement.

### II—Data Capture and Formation of the 3D Point Cloud:

At this stage, the TrueDepth technology will project a grid of infrared points over the patient's scalp, while the infrared camera captures the pattern, measuring the distortion of the points. By combining the depth data obtained with the RGB image captured by the standard camera, TrueDepth technology will construct a 3D mesh or a point cloud representing the anatomy of the scanned region of the patient's scalp.

### III—Generation of the 3D Mesh (“3D MESH”);

As the points are meticulously marked, the application will delimit, paint, and automatically record the region, generating a more detailed mesh model of the head (constructed from the data collected by TrueDepth), displaying detailed and continuous visualizations of the scanned surface of the scalp.

This mesh is referred to as the 3D mesh (“3D MESH”) and will be generated from this point cloud, where algorithms and processing techniques are applied to connect adjacent points and form triangular or polygonal faces. These connected faces create a continuous and detailed visual representation of the scanned surface of the scalp. The 3D mesh (“3D MESH”) consists of vertices, edges, and faces that define the geometry and topology of the scalp. Each vertex represents a specific point in three-dimensional space, and the edges and faces connect these vertices to form a continuous surface that accurately represents the detailed characteristics of the patient's scalp.

### IV—Area Measurement:

Area measurement is performed through tactile interaction with the desired region, simply by sliding the finger over the 3D model generated by the application, so that the selected area of the 3D mesh is measured. This mesh is composed of micropolygons, allowing precise calculation of surface area, even in regions with irregular curvature. The measurement is performed by the application by summing the areas of the micropolygons of the demarcated regions. This measurement method reduces distortions caused by the irregularity and curvature of the scalp's surface, providing a high degree of accuracy in measuring bald areas, as the 3D model compensates for the distortions and irregularities of the scalp's surface.

Since the distances in the model are valued over the 3D mesh, the area is calculated on the surface of the head, resulting in a highly precise measurement of areas without hair.

In one of the ideal applications of the present invention, the measurement of bald areas (recipient areas), areas with thin hair, and areas with thick hair (donor areas) will be performed through the use of a system that includes the use of a mobile app or other software for processing 3D models, properly downloaded on a mobile phone or tablet or any electronic device designed for 3D mapping and reconstruction, equipped with Photogrammetry technology, which will capture images of the scalp. In an ideal application, the method of the present invention involves the use of Photogrammetry technology for measuring bald areas (recipient areas), areas with thinning hair, and areas with thick hair (donor areas) by measuring the surface of 3D models and/or 3D meshes (“3D MESH”) created by these technologies or other similar technologies that generate life-size 3D models, comprising the following steps:

### I—Device Activation and Scanning of the Scalp:

To perform the measurement, the operator will power on the electronic device equipped with Photogrammetry technology and open the measurement application/software. Subsequently, with the software running (FIG. 02), the operator, using the device's camera, will position the device in such a way that the surface of the patient's scalp is within the field of view of the Photogrammetry technology. With the application still open (FIG. 02), the operator, using the device's camera, will capture several images of the patient's scalp from different angles, ensuring complete coverage of the scalp's surface that the patient wishes to measure.

### II—Data Capture and Formation of the 3D Point Cloud:

At this point, after capturing the images, the Photogrammetry technology will analyze the photos, identifying and correlating key reference points between them. This analysis results in the construction of a 3D mesh or point cloud, which accurately represents the anatomy of the scanned scalp region of the patient.

### III—Generation of the 3D Mesh (“3D MESH”);

As the points are meticulously marked, the application will automatically delimit, color, and record the region, generating a more detailed mesh model of the head (constructed from the images collected by Photogrammetry), which displays continuous and detailed visualizations of the scanned scalp surface.

This mesh is called a 3D mesh (“3D MESH”), and will be generated from this point cloud, where algorithms and processing techniques are applied to connect adjacent points and form triangular or polygonal faces. These connected faces create a continuous and detailed visual representation of the scanned scalp surface.

The 3D mesh (“3D MESH”) consists of vertices, edges, and faces that define the geometry and topology of the scalp. Each vertex represents a specific point in three-dimensional space, and the edges and faces connect these vertices to form a continuous surface that accurately represents the features of the patient's scalp.

### IV—Area Measurement:

Area measurement is performed through tactile interaction with the desired region, simply sliding the finger over the 3D model generated by the application to measure the selected 3D mesh area. This mesh is composed of micropolygons, enabling precise surface area calculation, even in regions with irregular curvature. The measurement is carried out by the application through the summation of the areas of the micropolygons in the marked regions. This measurement method reduces distortions caused by the irregularity and curvature of the scalp surface, providing a high degree of precision in measuring bald areas, as the 3D model compensates for distortions and irregularities of the scalp surface.

Since distances in the model are valued over the 3D mesh, the area is calculated on the surface of the head, resulting in a highly precise measurement of hairless areas. Thus, the method proposed by this invention utilizes technologies equipped with three-dimensional information capture mechanisms, such as TrueDepth, Structured Light, LiDAR, Time-of-Flight (ToF) Sensors, Photogrammetry, Stereoscopic Vision, and similar technologies present in smartphones or tablets or any electronic devices intended for 3D mapping and reconstruction, along with applications or other image processing software for 3D models, to enable the measurement of bald areas (receiver areas), areas with thin hair, and areas with thick hair (donor areas) on the scalp with high accuracy. This provides an efficient and precise solution, reducing costs for the patient and promoting better results in hair implants.

In summary, the proposed method uses technologies equipped with three-dimensional information capture mechanisms, such as TrueDepth, Structured Light, LIDAR, Time-of-Flight (ToF) Sensors, Photogrammetry, Stereoscopic Vision, and similar technologies, to map and measure bald areas (receiver areas), areas with thin hair, and areas with thick hair (donor areas) on the scalp. Through a smartphone, tablet, or another electronic device with 3D mapping and reconstruction capabilities, and a specialized application or software, it is possible to capture images, mark the area to be measured, and obtain an accurate evaluation of the quantitative degree of baldness (bald area). This innovation provides an advanced technological approach for the diagnosis and treatment of baldness.

Although the scope of the invention has been described herein with reference to specific embodiments, this description should not be interpreted in a limiting sense. Various modifications of the disclosed embodiments, as well as alternative embodiments of the invention, will become apparent to those skilled in the art upon reference to the description of the invention.

Furthermore, various other embodiments may have different configurations, components, or procedures than those described herein. A person skilled in the art will therefore understand that certain aspects shown in the figures may not be necessary.

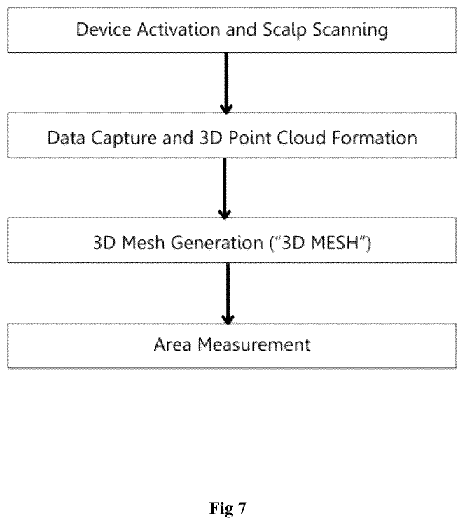

Experts in the field will appreciate that the steps shown in FIG. 07 can be modified in various ways. For example, the order of the steps can be rearranged; sub-steps can be executed in parallel; the steps shown can be omitted, or other steps can be included, etc.

From the previous description, it will be appreciated that specific embodiments have been described here for illustration purposes, but various modifications can be made to these embodiments, including combinations between them. Furthermore, although the advantages associated with certain embodiments have been described in the context of those embodiments, other embodiments may also exhibit such advantages, and not all embodiments need necessarily display such advantages. Therefore, the disclosure may include other embodiments not shown or described here.

Thus, it should be understood that the above description is intended to be illustrative and not restrictive. For example, the embodiments described above (and/or aspects thereof) can be used in combination with each other. Furthermore, many modifications can be made to adapt a particular situation or material to the teachings of the invention without departing from its scope. Although the dimensions, quantities, and types of materials described here are intended to define the parameters of the invention, they are in no way limiting but rather illustrative embodiments.

Many other embodiments will be apparent to those skilled in the art upon review of the above description. The scope of the invention should therefore be determined with reference to the appended claims, along with the full scope of equivalents to which those claims are entitled. In the appended claims, the terms “first”, “second”, and “third” are used merely as labels and are not intended to impose numerical requirements on their objects.

The present methods may involve any one or all of the steps or conditions discussed above in various combinations, as applicable. For example, it will be readily understood by one skilled in the art that, in some of the disclosed methods, certain steps may be excluded, or additional steps may be performed without altering the viability of the methods.

1. A method for Measuring the Surface of the Scalp for the Measurement of Bald Areas (recipient areas), Areas with Thinned Hair, Areas with Thick Hair (donor areas), using the counting of micropolygons in 3D models and/or 3D meshes created by technologies that generate life-size 3D models, the method comprising:
Receive the 3D model and/or 3D mesh through the use of technologies equipped with mechanisms for capturing three-dimensional information;
Delimit the area of interest through an interactive interface on the electronic device, directly on the virtual surface of the 3D model or 3D mesh, using any interactive interface designed to capture multiple input modalities;
Process the micropolygons within the selected region, identifying the micropolygons that are fully or partially included in the demarcated area;
Count the micropolygons to measure the area, determining the total area through the cumulative sum of the individual areas of the micropolygons that make up the selected region.

2. A method for Scalp Surface Mapping to Measure Bald Areas (recipient areas), Areas with Thinned Hair, Areas with Thick Hair (donor areas), characterized by the use of TrueDepth, Structured Light, LiDAR, Time-of-Flight (ToF) Sensors, Photogrammetry, Stereoscopic Vision, and similar technologies, for measuring bald areas (recipient areas), areas with thinned hair, areas with thick hair (donor areas), through the measurement of the surface of 3D models and/or 3D meshes created by these technologies or other similar technologies that generate life-size 3D models.

3. A method for Scalp Surface Mapping to Measure Bald Areas (recipient areas), Areas with Thinned Hair, Areas with Thick Hair (donor areas), characterized by the use of technologies equipped with mechanisms for capturing three-dimensional information, which will be used to generate a 3D mesh (“3D MESH”), serving as the foundation for rendering the 3D model of the patient's scalp for the mapping and measurement of bald areas (recipient areas), areas with thinned hair, areas with thick hair (donor areas).

4. The method for Scalp Surface Mapping to Measure Bald Areas (recipient areas), Areas with Thinned Hair, Areas with Thick Hair (donor areas), using the counting of micropolygons in 3D models and/or 3D meshes created by technologies that generate life-size 3D models, according to claim 2, characterized by the method comprising:
Activate the electronic device and scan the scalp using a specialized measurement application;
Capture data collected through a technology that generates life-size 3D models and form the 3D Point Cloud;
Generate the 3D Mesh (“3D MESH”) by marking points, by the operator, through the application, delimiting, painting, and automatically recording the scalp region, generating a more detailed mesh model of the head, created based on data from the technology that generates 3D models;
Measure the area by counting micropolygons in 3D models and/or 3D meshes created by technologies that generate life-size 3D models.

5. The method for Scalp Surface Mapping to Measure Bald Areas (recipient areas), Areas with Thinned Hair, Areas with Thick Hair (donor areas), using the counting of micropolygons in 3D models and/or 3D meshes created by technologies that generate life-size 3D models, according to claim 4, characterized by the step of Activating the device and scanning the scalp comprising the operator turning on the electronic device equipped with three-dimensional information capture technology; opening the mapping, processing, and image reconstruction application/software for specialized 3D measurement; with the software running, using the device's camera, positioning the electronic device so that the surface of the patient's scalp is within the technology's field of view; capturing multiple images of the patient's scalp from different angles, using the device's camera, ensuring full coverage of the patient's scalp surface that is to be measured.

6. The method for Scalp Surface Mapping to Measure Bald Areas (recipient areas), Areas with Thinned Hair, Areas with Thick Hair (donor areas), according to claim 5, characterized by the electronic device equipped with three-dimensional information capture technology being, preferably, a mobile phone or tablet.

7. The method for Scalp Surface Mapping to Measure Bald Areas (recipient areas), Areas with Thinned Hair, Areas with Thick Hair (donor areas), according to claim 4, characterized by the step of Data Capture and Formation of the 3D Point Cloud comprising the analysis, by one of the technologies equipped with three-dimensional information capture mechanisms, of the captured images, identifying and correlating singular reference points that are matched across the various images to compute the three-dimensional spatial coordinates of the scalp, resulting in the construction of a 3D mesh or a 3D point cloud, which accurately represents the anatomy of the scanned region of the patient's scalp.

8. The method for Scalp Surface Mapping to Measure Bald Areas (recipient areas), Areas with Thinned Hair, Areas with Thick Hair (donor areas), according to claim 4, characterized by the step of Generation of the 3D Mesh (“3D MESH”) being the result of the analysis, identification, and correlation of the captured images, accurately representing the anatomy of the scanned region of the patient's scalp.

9. The method for Scalp Surface Mapping to Measure Bald Areas (recipient areas), Areas with Thinned Hair, Areas with Thick Hair (donor areas), according to claim 4, characterized by the step of Generation of the 3D Mesh (“3D MESH”) comprising the meticulous marking of points, delimitation, painting, and automatic recording of the region, performed by the application/software, generating a more detailed 3D mesh model of the head, which displays detailed and continuous visualizations of the scanned scalp surface.

10. The method for Scalp Surface Mapping to Measure Bald Areas (recipient areas), Areas with Thinned Hair, Areas with Thick Hair (donor areas), according to claim 4, characterized by the step of Generation of the 3D Mesh (“3D MESH”) comprising the meticulous marking of points, delimitation, painting, and automatic recording of the region, performed by the application/software, generating a more detailed 3D mesh model of the head, which displays detailed and continuous visualizations of the scanned scalp surface.

11. The method for Scalp Surface Mapping to Measure Bald Areas (recipient areas), Areas with Thinned Hair, Areas with Thick Hair (donor areas), according to claim 10, characterized by the 3D mesh (“3D MESH”) being generated from the point cloud, where algorithms and processing techniques are applied to connect adjacent points and form connected triangular or polygonal faces that create a continuous and detailed visual representation of the scanned scalp surface.

12. The method for Scalp Surface Mapping to Measure Bald Areas (recipient areas), Areas with Thinned Hair, Areas with Thick Hair (donor areas), according to claim 11, characterized by the 3D mesh (“3D MESH”) consisting of vertices, edges, and faces that define the geometry and topology of the scalp, where each vertex represents a specific point in three-dimensional space, and the edges and faces connect these vertices to form a continuous surface that accurately represents the characteristics of the patient's scalp.

13. The method for Scalp Surface Mapping to Measure Bald Areas (recipient areas), Areas with Thinned Hair, Areas with Thick Hair (donor areas), according to claim 4, characterized by the step of Area Measurement being performed by the application, through the sum of the areas of the micropolygons of the demarcated regions, carried out through an interactive interface designed to capture multiple inputs, on the 3D model generated by the application so that the area of the selected 3D mesh is measured.

14. Method for Scalp Surface Mapping to Measure Bald Areas (recipient areas), Areas with Thinned Hair, Areas with Thick Hair (donor areas), according to claim 13, characterized by the 3D mesh being composed of micropolygons that allow for the precise calculation of the surface area, even in regions with irregular curvature, mitigating distortions caused by the irregularity and curvature of the scalp surface, providing a degree of accuracy in measuring the bald areas, as the 3D model compensates for the distortions and irregularities of the scalp surface, resulting in a highly accurate measurement of hairless areas.

15. Scalp Surface Measurement System for Measuring Bald Areas (recipient areas), Areas with Thinned Hair, Areas with Thick Hair (donor areas), using the counting of micropolygons in 3D models and/or 3D meshes created by technologies that generate life-size 3D models, characterized by, in an ideal application, comprising a camera hardware equipped with at least one three-dimensional information capture technology, an electronic device, and a measurement application or software installed on the electronic device.

16. Computer Program for Scalp Surface Measurement to Measure Bald Areas (recipient areas), Areas with Thinned Hair, Areas with Thick Hair (donor areas), using the counting of micropolygons in 3D models and/or 3D meshes created by technologies that generate life-size 3D models, configured to:
Capture three-dimensional information of the scalp;
Receive digital image data collected by technology equipped with three-dimensional information capture mechanisms;
Analyze the captured images, identifying and correlating reference points that are matched across various images to compute the three-dimensional spatial coordinates of the scalp;
Construct a 3D point cloud, which accurately represents the geometry and topography of the scanned scalp;
Delimit, paint, and automatically record the region, generating a 3D mesh (3D MESH), which displays detailed and continuous visualizations of the scanned scalp surface;
Create, from the 3D mesh (3D MESH), a continuous and detailed visual representation of the scanned scalp surface;
Measure the area of the scalp through tactile interaction in the desired region;
Display, for viewing through the electronic device, a simulated image of the scalp.
